(19) United States
(12) Patent Application Publication
(10) Pub. No.: US 2008/0086006 A1
(43) Pub. Date: Apr. 10, 2008

(54) Process for Making Substituted Piperidines

(76) Inventor: Todd D. Nelson, (US)

Correspondence Address: MERCK AND CO., INC, P O BOX 2000, RAHWAY, NJ, 07065-0907, US

(21) Appl. No.: 11/793,944

(22) PCT Filed: Dec. 21, 2005
(86) PCT No.: PCT/US05/46718

§ 371 Date: Dec. 3, 2007

Related U.S. Application Data
(60) Provisional application No. 60/638,157, filed on Dec. 22, 2004.

(51) Int. Cl. C07F 7/08; C07D 211/82; C07F 15/00

(52) U.S. Cl. 546/236; 546/339; 556/12; 546/2; 556/14

(57) ABSTRACT

The present invention provides a process for the preparation of substituted piperidines which comprises an asymmetric hydrogenation of vinyl fluoride in the presence of a metal precursor complexed with a chiral mono- or biphosphine ligand.

### FIELD OF THE INVENTION

The present invention relates to methods of making substituted piperidines. The processes comprise an asymmetric hydrogenation of vinyl fluoride in the presence of a metal precursor complexed with a chiral mono- or biphosphine ligand. In particular, this invention is directed to methods for making N-benzyl 3-fluoro substituted piperidines useful as constituents of drug candidates and in the synthesis of other biologically active molecules.

### SUMMARY OF THE INVENTION

The present invention concerns a process for the preparation of derivatives of Formula I. The process utilizes an asymmetric hydrogenation of a vinyl fluoride or derivative thereof, in the presence of a metal precursor complexed with a chiral mono- or bisphosphine ligand. The process of the present invention is applicable to the preparation of benzyl fluoro-substituted piperidine derivatives on a pilot plant or industrial scale. The derived benzyl fluoro-substituted piperidines are useful as constituents of drug candidates or to prepare a wide variety of other biologically active molecules.

The instant invention further encompasses certain intermediate compounds.

### DETAILED DESCRIPTION OF THE INVENTION

The present invention provides a process for making a compound of Formula (I): 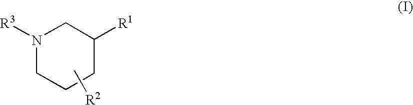
or a pharmaceutically acceptable salt thereof,
wherein
R¹ is halogen, oxygen, CONH₂, nitrogen, sulfur, silicon, optionally substituted C₁-C₆ alkyl or optionally substituted aryl;
R² is oxygen, amino, halogen, CONH₂, nitrogen, sulfur, or C₀-C₄ alkyl optionally substituted with one or more groups selected from hydrogen, hydroxy, amino, and amino-heteroaryl;
R³ is sulfur, optionally substituted C₁-C₆ alkyl, aryl, phosphorous, silicon, benzyl, CBZ, carbamate, C₁-C₆ alkyl-optionally substituted aryl, or C(═O)O-optionally substituted aryl;
the process comprising an asymmetric reduction of a compound of Formula (B): 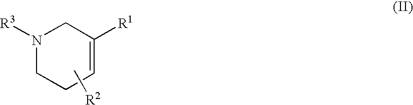
wherein
R¹, R² and R³ each is as defined above,
in a suitable organic solvent in the presence of a metal precursor complexed to a chiral mono- or bisphosphine ligand.

In a first aspect of the inventive process, the present invention provides a process for making a compound of Formula (I), or a pharmaceutically acceptable salt thereof, wherein

R¹ is halogen or optionally substituted aryl.

In a second aspect, the present invention provides a process for making a compound of Formula (I), or a pharmaceutically acceptable salt thereof, wherein

R¹ is halogen.

In third aspect, the present invention provides a process for making a compound of Formula (I), or a pharmaceutically acceptable salt thereof, wherein

R¹ is fluorine.

In a fourth aspect, the present invention provides a process for making a compound of Formula (I), or a pharmaceutically acceptable salt thereof, wherein

R¹ is optionally substituted aryl.

In a fifth aspect, the present invention provides a process for making a compound of Formula (I), or a pharmaceutically acceptable salt thereof, wherein

R² is C₀-C₄ alkyl optionally substituted with hydroxyl, amino or amino-heteroaryl.

In a sixth aspect, the present invention provides a process for making a compound of Formula (I), or a pharmaceutically acceptable salt thereof, wherein

R³ is C₁-C₆-optionally substituted aryl.

In a seventh aspect, the present invention provides a process for making a compound of Formula (I), or a pharmaceutically acceptable salt thereof, wherein

R³ is benzyl.

In an eighth aspect of the inventive process, the present invention provides a process for making a compound of Formula (I), or a pharmaceutically acceptable salt thereof, wherein

the metal precursor is a rhodium precursor.

In an embodiment of this eighth aspect, the present invention provides a process for making a compound of Formula (I), or a pharmaceutically acceptable salt thereof, wherein

the metal precursor is [Rh(cod)Cl]₂.

In a ninth aspect of the inventive process, the present invention provides a process for making a compound of Formula (I), or a pharmaceutically acceptable salt thereof, wherein

the organic solvent is methanol.

In a tenth aspect of the inventive process, the present invention provides a process for making a compound of Formula (I), or a pharmaceutically acceptable salt thereof, wherein

the organic solvent is ethanol or isopropyl alcohol.

In an eleventh aspect of the inventive process, the present invention provides a process for making a compound of Formula (I), or a pharmaceutically acceptable salt thereof, wherein

the chiral bisphosphine ligand is a ferrocenyl bisphosphine ligand of the structural formula: 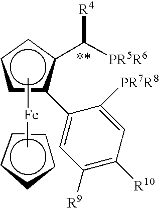
wherein ** is a carbon stereogenic center with an (R)-configuration;
R⁴ is C₁-C₄ alkyl or aryl;
R⁵, R⁶, R⁷ and R⁸ are each independently C₁-C₆ alkyl, C₅₋₁₂ cycloalkyl, heteroaryl or aryl, wherein said aryl and heteroaryl is optionally substituted with one or more C₁-C₆ fluoroalkyl, halogen, C₁-C₄ alkyl, CF₃, or O—C₁-C₄ alkyl; and R⁹ and R¹⁰ are each independently halogen, hydrogen, C₁-C₆ alkyl, C₁-C₆ fluoroalkyl, C₅-C₁₂ cycloalkyl or C₁-C₄ alkoxy.

The present invention further provides an intermediate compound of Formula (III), or an organic acid or metal acid thereof:

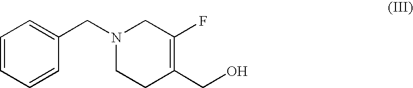

The process of the present invention contemplates that, where a rhodium metal precursor is used, the catalytic complex of the rhodium metal precursor and the chiral phosphine ligand may be either (a) generated in situ by the sequential or contemporaneous addition of the rhodium metal precursor and chiral phosphine ligand to the reaction mixture or (b) pre-formed with or without isolation and then added to the reaction mixture. Pre-formed catalytic complexes are represented by the below formulas, where (R′)₂P—P(R)₂ represents either a chelating chiral bidentate biphosphine ligand or two non-chelating chiral monodentate phosphine ligands, X represents a non-coordinating anion, such as trifluoromethanesulfonate, tetrafluoroborate, and hexafluorophosphate, and L is a neutral ligand such as an olefin (or chelating di-olefin such as 1,5-cyclooctadiene or norbornadiene) or a solvent molecule (such as MeOH and TFE): 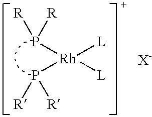
In the case where olefin is arene, the complex is represented by the formula:

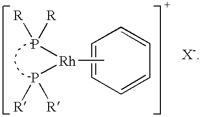

The pre-formed catalytic complex in the case where X represents halogen is represented by the formula:

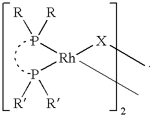

In one embodiment of the process of the present invention, the chiral phosphine ligand has the following structural formula: 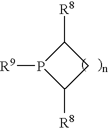
wherein n is 1, 2, or 3; R⁸ is C₁₋₈ alkyl or C₆₋₁₀ aryl; and R⁹ is aryl or a ferrocenyl phospholane radical.

In one class of this embodiment, R⁹ is phenyl and R⁸ is C₁₋₄ alkyl or aryl.

A second class of this first embodiment encompasses the FerroLANE, FerroTANE, PhenylLANE, and PhenylTANE series having the following structural formulae: 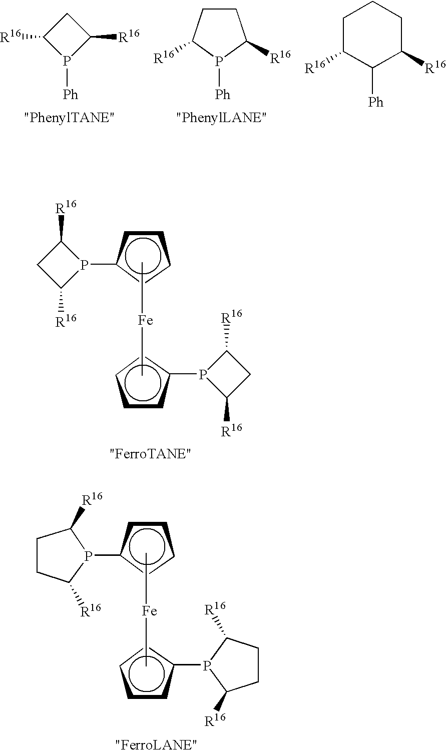
wherein R¹⁶ is C₁₋₄ alkyl or aryl;
or the corresponding enantiomers thereof.

In a second embodiment of the process of the present invention, the chiral bisphosphine ligand has the following structural formula: 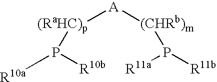
wherein m and p are each 0 or 1;
R^{a} and R^{b} are each independently hydrogen, C₁₋₄ alkyl, or C₃₋₆ cycloalkyl;
A represents (a) a C₁₋₅ alkylene bridge optionally containing one to two double bonds said C₁₋₅ alkylene bridge being unsubstituted or substituted with one to four substituents independently selected from the group consisting of C₁₋₄ alkyl, C₁₋₄ alkoxy, aryl, and C₃₋₆ cycloalkyl and said C₁₋₅ alkylene bridge being optionally fused with two C₅₋₆ cycloalkyl, C₆₋₁₀ aryl, or C₆₋₁₀ heteroaryl groups unsubstituted or substituted with one to four substituents independently selected from the group consisting of C₁₋₄ alkyl, C₁₋₄ alkoxy, chloro, and fluoro; (b) a 1,2-C₃₋₈ cycloalkylene bridge optionally containing one to three double bonds and one to two heteroatoms selected from NC₀₋₄ alkyl, N(CH₂)₀₋₁Ph, NCOC₁₋₄ alkyl, NCOOC₁₋₄ alkyl, oxygen, and sulfur and said 1,2-C₃₋₈ cycloalkylene bridge being unsubstituted or substituted with one to four substituents independently selected from the group consisting of C₁₋₄ alkyl, C₁₋₄ alkoxy, oxo, aryl, and C₃₋₆ cycloalkyl; (c) a 1,3-C₃₋₈ cycloalkylene bridge optionally containing one to three double bonds and one to two heteroatoms selected from NC0-4 alkyl, N(CH2)0-1Ph, NCOC1-4 alkyl, NCOOC1-4 alkyl, oxygen, and sulfur and said 1,3-C₃₋₈ cycloalkylene bridge being unsubstituted or substituted with one to four substituents independently selected from the group consisting of C1-4 alkyl, C1-4 alkoxy, oxo, aryl, and C₃₋₆ cycloalkyl; or (d) 1,2-phenylene unsubstituted or substituted with one to three substituents independently selected from halogen, C1-4 alkyl, hydroxy, and C1-4 alkoxy; and R10a, R10b, R11a, and R11b are each independently C1-6 alkyl, C3-6 cycloalkyl, or aryl with alkyl, cycloalkyl, and aryl being unsubstituted or substituted with one to three groups independently selected from the group consisting of C₁₋₄ alkyl, C₁₋₄ alkoxy, chloro, and fluoro; or R^{10a} and R^{10b} when taken together or R^{11a} and R^{11b} when taken together can form a 4- to 7-membered cyclic aliphatic ring unsubstituted or substituted with two to four substituents independently selected from the group consisting of C₁₋₄ alkyl, C₁₋₄ alkoxy, hydroxymethyl, C₁₋₄ alkoxymethyl, aryl, and C₃₋₆ cycloalkyl and said cyclic aliphatic ring being optionally fused with one or two aryl groups;

In one class of this embodiment, R10a and R10b represent the same substituent which are both structurally distinct from R11a and R11b which represent the same but structurally distinct substituent. In a subclass of this class, R10a and R10b are both optionally substituted C1-6 alkyl, and R11a and R11b are both optionally substituted C3-6 cycloalkyl. In a second subclass of this class, R10a and R10b are both optionally substituted aryl, and R11a and R11b are both optionally substituted C3-6 cycloalkyl. In a third subclass of this class, R10a and R10b are both substituted aryl, and R11a and R11b are both unsubstituted aryl. In a fourth subclass of this class, R10a and R10b are both optionally substituted C1-6 alkyl, and R11a and R11b are both optionally substituted aryl.

A second class of this second embodiment encompasses chiral bisphosphine ligands disclosed in U.S. Pat. No. 4,994,615, the contents of which are incorporated by reference herein in their entirety. Non-limiting embodiments of this class of chiral 1,4-bisphosphine ligands are represented by structural formulae: 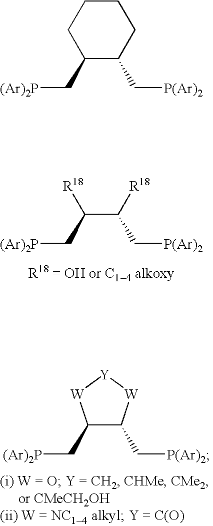
or the corresponding enantiomers thereof.

Representative, but non-limiting, specific embodiments of this class of chiral bisphosphine ligands are the following structures: 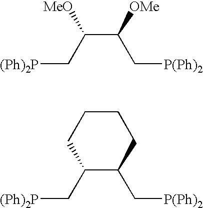
or the corresponding enantiomers thereof.

A third class of this second embodiment encompasses chiral bisphosphine ligands disclosed in U.S. Pat. Nos. 5,008,457; 5,171,892; 5,206,398; 5,329,015; 5,532,395; 5,386,061; 5,559,267; 5,596,114; and 6,492,544, the contents of all of which are incorporated by reference herein in their entirety. Non-limiting embodiments of this class of chiral bisphosphine ligands are represented by:

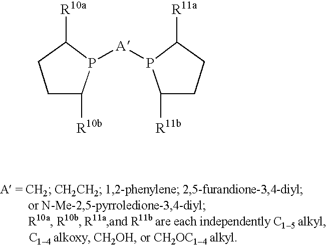

Representative, but non-limiting, specific embodiments of this class of chiral bisphosphine ligands are the following structures: 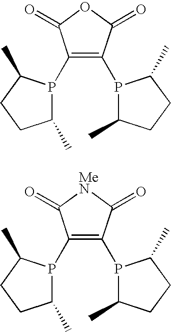
or the corresponding enantiomers thereof.

A fourth class of this second embodiment encompasses bisphosphine ligands of the structural formula: 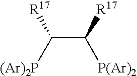
wherein Ar is aryl and R¹⁷ is C₁₋₄ alkyl or aryl;
or the corresponding enantiomers thereof;
with the proviso that when Ar is unsubstituted phenyl, R¹⁷ is not methyl.

A third embodiment of the chiral bisphosphine ligand encompasses biaryl or biheteroaryl bisphosphine ligands of the structural formulae: 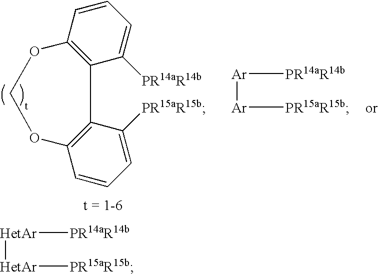
wherein Ar is phenyl or naphthyl unsubstituted or substituted with one to four substituents independently selected from C₁₋₄ alkyl, C₁₋₄ alkoxy, chloro, and fluoro; or two adjacent substituents on Ar together with the carbon atoms to which they are attached form a five-membered methylenedioxy ring;
HetAr is pyridyl or thienyl each of which is unsubstituted or substituted with one to four substituents independently selected from C₁₋₄ alkyl, C₁₋₄ alkoxy, chloro, and fluoro; or two adjacent substituents on HetAr together with the carbon atoms to which they are attached form a five-membered methylenedioxy ring;
R^{14a}, R^{14b}, R^{15a}, and R^{15b} are each independently C₁₋₄ alkyl, aryl, or C₃₋₆ cycloalkyl wherein aryl and cycloalkyl are unsubstituted or substituted with one to four substituents independently selected from C₁₋₄ alkyl and C₁₋₄ alkoxy; or
or R^{14a} and R^{14b} when taken together or R^{15a} and R^{15b} when taken together can form a 4- to 7-membered cyclic aliphatic ring unsubstituted or substituted with two to four substituents independently selected from the group consisting of C₁₋₄ alkyl,
C₁₋₄ alkoxy, hydroxymethyl, C₁₋₄ alkoxymethyl, aryl, and C₃₋₆ cycloalkyl and said cyclic aliphatic ring being optionally fused with one or two aryl groups.

In one class of this embodiment, R14a and R14b represent the same substituent which are both structurally distinct from R^{15a} and R^{15b} which represent the same but structurally distinct substituent. In a subclass of this class, R14a and R14b are both optionally substituted C₁₋₆ alkyl, and R^{15a} and R^{15b} are both optionally substituted C₃₋₆ cycloalkyl. In a second subclass of this class, R^{14a} and R^{14b} are both optionally substituted aryl, and R^{15a} and R^{15b} are both optionally substituted C₃₋₆ cycloalkyl. In a third subclass of this class, R^{14a} and R^{14b} are both substituted aryl, and R^{15a} and R^{15b} are both unsubstituted aryl. In a fourth subclass of this class, R^{14a} and R^{14b} are both optionally substituted C₁₋₆ alkyl, and R^{15a} and R^{15b} are both optionally substituted aryl.

Representative, but non-limiting, examples of this third embodiment of chiral bisphosphine ligands are the following structures: 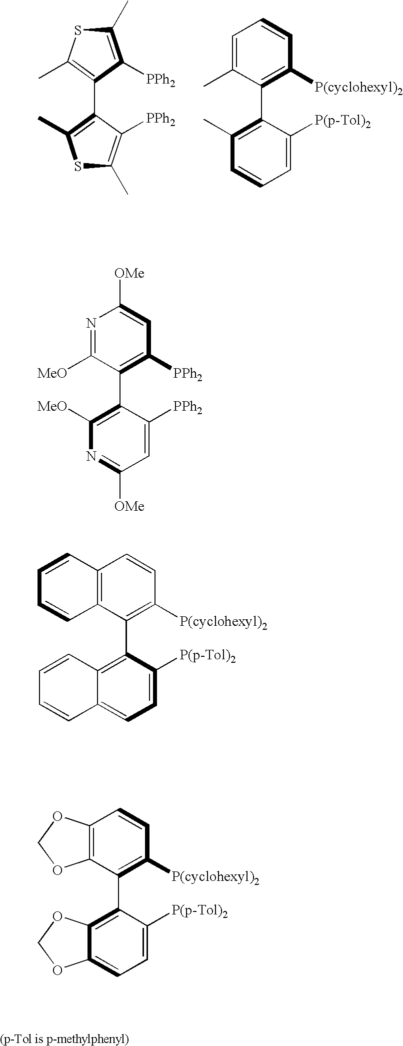
or the corresponding enantiomers thereof.

A fourth embodiment encompasses chiral bisphosphine ligands disclosed in U.S. Pat. Nos. 5,874,629 and 6,043,387, the contents of both of which are incorporated by reference herein in their entirety. Non-limiting sub-embodiments of this embodiment of chiral bisphosphine ligands are represented by: 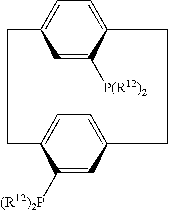
R¹²=C₁₋₄ alkyl, C₃₋₆ cycloalkyl, or aryl
   or the corresponding enantiomers thereof.

A specific, but non-limiting, example of this embodiment of bisphosphine ligands is the following compound: 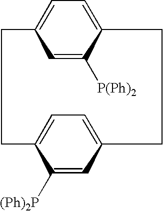
or the corresponding enantiomer thereof.

In a fifth embodiment of the process of the present invention, the chiral bisphosphine ligand has the following structural formula: 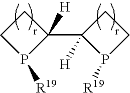
wherein r is 1, 2, or 3; and R¹⁹ is C₁₋₄ alkyl or aryl;
or the corresponding enantiomers thereof.

A specific, but non-limiting, example of this embodiment of chiral bisphosphine ligands is the following: 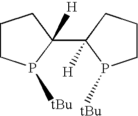
or the corresponding enantiomer thereof.

In a sixth embodiment of the process of the present invention, the chiral phosphine ligand is of the structural formula: 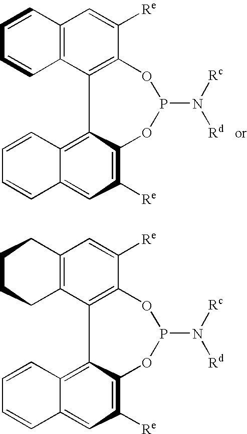
wherein R^{e} is hydrogen or methyl; R^{c} and R^{d} are each independently hydrogen, C₁₋₄ alkyl, benzyl, or α-methylbenzyl; or R^{c} and R^{d} together with the nitrogen atom to which they are attached form a pyrrolidine or piperidine ring.

In a seventh embodiment of the process of the present invention, the chiral bisphosphine ligand is a ferrocenyl bisphosphine ligand of the structural formula: 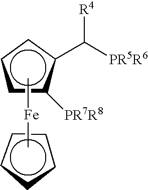
wherein R⁴ is C₁₋₄ alkyl or aryl; and
R⁵, R⁶, R⁷ and R⁸ are each independently C₁-C₆ alkyl, C₅₋₁₂ cycloalkyl, heteroaryl or aryl, wherein said aryl and heteroaryl is optionally substituted with one or more C₁-C₆ fluoroalkyl, halogen, C₁-C₄ alkyl, CF₃, or O—C₁-C₄ alkyl.

In a class of this seventh embodiment, R⁴ is methyl; R⁵, R⁶, R⁷ and R⁸ are each independently C₁-C₆ alkyl or phenyl, wherein said phenyl is optionally substituted with one or more C₁-C₄ alkyl. In a subclass of this class, R⁴ is methyl; R⁵, R⁶ are each independently C₁-C₄ alkyl; and R⁷ and R⁸ are each independently phenyl. In an additional subclass of this class, R⁴ is methyl; R⁵, R⁶ are each independently phenyl, substituted with methyl; and R⁷ and R⁸ are each independently C₁-C₄ alkyl. In a further subclass of this class, R⁴ is methyl; R⁵, R⁶ are each independently phenyl substituted with two methyl groups; and R⁷ and R⁸ are each independently C₁-C₄ alkyl.

In an eighth embodiment of the process of the present invention, the chiral bisphosphine ligand is a ferrocenyl bisphosphine ligand of the structural formula: 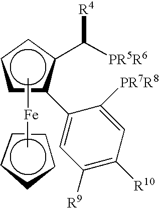
wherein R⁴ is C₁-C₄ alkyl or aryl;
R⁵, R⁶, R⁷ and R⁸ are each independently C₁-C₆ alkyl, C₅₋₁₂ cycloalkyl, heteroaryl or aryl, wherein said aryl and heteroaryl is optionally substituted with one or more C₁-C₆ fluoroalkyl, halogen, C₁-C₄ alkyl, CF₃, or O—C₁-C₄ alkyl; and R⁹ and R¹⁰ are each independently halogen, hydrogen, C₁-C₆ alkyl, C₁-C₆ fluoroalkyl, C₅-C₁₂ cycloalkyl or C₁-C₄ alkoxy. In a class of this eighth embodiment, R⁴ is methyl; R⁵, R⁶, R⁷ and R⁸ are each independently cyclohexyl or phenyl, wherein said phenyl is optionally substituted with one or more C₁-C₄ alkyl, CF₃, or O—C₁-C₄ alkyl; and R⁹ and R¹⁰ are each independently hydrogen. In a subclass of this class, R⁴ is methyl; R⁵ and R⁶ are each independently cyclohexyl; R⁷ and R⁸ are each independently phenyl; and R⁹ and R¹⁰ are each independently hydrogen.

In an additional class of this eighth embodiment the carbon stereogenic center marked with an ** has the (R)-configuration as depicted in the structural formula:

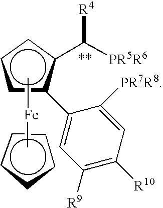

In a subclass of this class, R⁴ is methyl; R⁵, R⁶, R⁷ and R⁸ are each independently aryl or C₅₋₁₂ cycloalkyl; and R⁹ and R¹⁰ are each independently halogen or hydrogen. In a subclass of this subclass, R⁴ is methyl; R⁵, R⁶, R⁷ and R⁸ are each independently cyclohexyl or phenyl; and R⁹ and R¹⁰ are each independently hydrogen. In an additional subclass of this subclass, R⁴ is methyl; R⁵ and R⁶ are each independently cyclohexyl; R⁷ and R⁸ are each independently phenyl; and R⁹ and R¹⁰ are each independently hydrogen.

Ligands encompassed within this eighth embodiment are also referred to herein as “Walphos” (commercially available from Solvias, Inc., Fort Lee, N.J. 07024). A Walphos ligand having the following substituents: R⁴ is methyl; R⁵ and R⁶ are each independently cyclohexyl; R⁷ and R⁸ are each independently phenyl; and R⁹ and R¹⁰ are each independently hydrogen, is referred to herein as Walphos (SL-W003-1).

Chiral ferrocenyl bisphosphine ligands encompassed within the process of the present invention are disclosed in U.S. Pat. Nos. 5,371,256; 5,463,097; 5,466,844; 5,563,308; 5,563,309; 5,565,594; 5,583,241; and RE37,344, the contents of all of which are incorporated by reference herein in their entirety.

The asymmetric hydrogenation reaction of the present invention is carried out in a suitable organic solvent. Suitable organic solvents include lower alkanols, such as methanol, ethanol, and isopropyl alcohol; 2,2,2-trifluoroethanol (TFE); hexafluoroisopropyl alcohol; phenol; fluorinated phenols; polyhydroxylated benzenes, such as 1,2,3-trihydroxybenzene (pyrogallol) and 1,2,3,4-tetrahydroxybenzene; tetrahydrofuran; dichloromethane; methyl t-butyl ether; and mixtures thereof.

The reaction temperature for the reaction may be in the range of about 10° C. to about 90° C. A temperature range for the reaction is about 40° C. to about 65° C.

The hydrogenation reaction can be performed at a hydrogen pressure range of about 0 psig to about 1500 psig. A hydrogen pressure range is about 80 psig to about 200 psig.

The rhodium metal precursor is [Rh(monoolefin)2Cl]2, [Rh(diolefin)Cl]2, [Rh(monoolefin)2acetylacetonate], [Rh(diolefin)acetylacetonate], [Rh(monoolefin)4]X, or [Rh(diolefin)2]X wherein X is a non-coordinating anion selected from the group consisting of methanesulfonate, trifluoromethanesulfonate (Tf), tetrafluoroborate (BF4), hexafluorophosphate (PF6), or hexafluoroantimonate (SbF6). In one embodiment the rhodium metal precursor is [Rh(cod)Cl]₂, [Rh(norbornadiene)Cl]₂, [Rh(cod)₂]X, or [Rh(norbornadiene)₂]X. In a class of this embodiment, the rhodium metal precursor is [Rh(cod)Cl]₂.

Throughout the instant application, unless otherwise indicated, these terms have the following meanings:

The term “% enantiomeric excess” (abbreviated “ee”) shall mean the % major enantiomer less the % minor enantiomer. Thus, a 70% enantiomeric excess corresponds to formation of 85% of one enantiomer and 15% of the other. The term “enantiomeric excess” is synonymous with the term “optical purity.”

The process of the present invention provides compounds of structural formula I with high optical purity, typically in excess of 50% ee. In one embodiment, compounds of formula I are obtained with an optical purity in excess of 70% ee. In a class of this embodiment, compounds of formula I are obtained with an optical purity in excess of 80% ee. In a subclass of this class, compounds of formula I are obtained with an optical purity in excess of 90% ee.

The term “enantioselective” shall mean a reaction in which one enantiomer is produced (or destroyed) more rapidly than the other, resulting in the predominance of the favored enantiomer in the mixture of products.

The alkyl groups specified above are intended to include those alkyl groups of the designated length in either a straight or branched configuration.

Exemplary of such alkyl groups are methyl, ethyl, propyl, isopropyl, butyl, sec-butyl, tertiary butyl, pentyl, isopentyl, hexyl, isohexyl, and the like. The term “C₀-C₆alkyl” includes alkyls containing 6, 5, 4, 3, 2, 1, or no carbon atoms. An alkyl with no carbon atoms is a hydrogen atom substituent when the alkyl is a terminal group and is a direct bond when the alkyl is a bridging group. The alkyl groups are unsubstituted or substituted with one to three groups independently selected from the group consisting of halogen, hydroxy, carboxy, aminocarbonyl, amino, C₁-C₄ alkoxy, and C₁₋₄ alkylthio.

The term “cycloalkyl” is intended to mean cyclic rings of alkanes of five to twelve total carbon atoms, or any number within this range (i.e., cyclopentyl, cyclohexyl, cycloheptyl, etc).

The term “C₁₋₅ alkylene” is intended to mean a methylene (—CH₂—), ethylene (—CH₂CH₂—), propylene (—CH₂CH₂CH₂—), butylene (—CH₂CH₂CH₂CH₂—), or a pentylene (—CH₂CH₂CH₂CH₂CH₂—) group.

The term “1,2-phenylene” is intended to mean a phenyl group substituted at the 1- and 2-positions.

The term “1,2-C₃₋₈ cycloalkylene” is intended to mean a cycloalkyl group of 3- to 8-carbons which is substituted at adjacent carbons of the ring, as exemplified by 1,2-disubstituted cyclohexyl and 1,2-disubstituted cyclopentyl. The cycloalkylene group is also intended to encompass a bicyclic ring system containing one pair of bridgehead carbon atoms, such as a bicyclo[2.2.1]heptyl ring system (exemplified by norbornane and norbornene) and a bicyclo[2.2.2]octyl ring system.

The term “1,3-C₃₋₈ cycloalkylene” is intended to mean a cycloalkyl group of 3- to 8-carbons which is substituted at the 1- and 3-positions of the cyclic ring system, as exemplified by 1,3-disubstituted cyclohexyl and 1,3-disubstituted cyclopentyl.

The term “halogen” is intended to include the halogen atoms fluorine, chlorine, bromine, and iodine.

The term “olefin” refers to a acyclic or cyclic hydrocarbon containing one or more double bonds including aromatic cyclic hydrocarbons. The term includes, but is not limited to, 1,5-cyclooctadiene (“cod”) and norbornadiene (“nbd”).

The abbreviation “cod” means “1,5-cyclooctadiene.”

The term “aryl” includes phenyl or naphthyl. Unless specified, “aryl” is unsubstituted or substituted with one to five substituents independently selected from phenyl, halogen, hydroxy, amino, carboxy, C₁₋₄ alkyl, C₁₋₄ alkoxy, C₁₋₄ alkylthio, C₁₋₄ alkylsulfonyl, and C₁₋₄ alkyloxycarbonyl, wherein the alkyl moiety of each is unsubstituted or substituted with one to five fluorines.

The term “heteroaryl” means a 5- or 6-membered aromatic heterocycle that contains at least one ring heteroatom selected from O, S and N. Heteroaryls also include heteroaryls fused to other kinds of rings, such as aryls, cycloalkyls and heterocycles that are not aromatic. Examples of heteroaryl groups include, but are not limited to, pyrrolyl, isoxazolyl, isothiazolyl, pyrazolyl, pyridinyl, oxazolyl, 1,2,4-oxadiazolyl, 1,3,4-oxadiazolyl, thiadiazolyl, thiazolyl, imidazolyl, triazolyl, tetrazolyl, furyl, triazinyl, thienyl, pyrimidinyl, pyrazinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, and dibenzofuranyl. “Heteroaryl” is unsubstituted or substituted with one to five substituents independently selected from fluoro, hydroxy, trifluoromethyl, amino, C₁₋₄ alkyl, and C₁₋₄ alkoxy.

The term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH— if a bridging group and a —NH₂ if a terminal group. Analogous bridging or terminal groups are clear for an O or S heteroatom.

The term “amine,” unless specifically stated otherwise, includes primary, secondary and tertiary amines.

The term “carbonyl,” unless specifically stated otherwise, includes a C₀₋₆alkyl substituent group when the carbonyl is terminal.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the alkyl and the aryl groups are optionally substituted. If only one of the multiple moieties is optionally substituted then it will be specifically recited such as “an alkylaryl, the aryl optionally substituted with halogen or hydroxyl.”

Compounds described herein may contain one or more double bonds and may thus give rise to cis/trans isomers as well as other conformational isomers. The present invention includes all such possible isomers as well as mixtures of such isomers unless specifically stated otherwise.

Compounds described herein can contain one or more asymmetric centers and may thus give rise to diastereoisomers and optical isomers. The present invention includes all such possible diastereoisomers as well as their racemic mixtures, their substantially pure resolved enantiomers, all possible geometric isomers, and pharmaceutically acceptable salts thereof. The above chemical Formulas are shown without a definitive stereochemistry at certain positions. The present invention includes all stereoisomers of the chemical Formulas and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The abbreviations used herein have the following tabulated meanings. Abbreviations not tabulated below have their meanings as commonly used unless specifically stated otherwise.

| | |
|---|---|
| Ac | Acetyl |
| Bn | Benzyl |
| CAMP | cyclic adenosine-3′,5′-monophosphate |
| DBU | 1,8-diazabicyclo[5.4.0]undec-7-ene |
| DMF | N,N-dimethylformamide |
| GC | gas chromatography |
| HPLC | high performance liquid chromatography |
| IPAC or IPAc | Isopropyl acetate |
| m-CPBA | metachloroperbenzoic acid |
| Ms | methanesulfonyl = mesyl = SO₂Me |
| Ms0 | methanesulfonate = mesylate |
| o-Tol | ortho-tolyl |
| PCC | pyridinium chlorochromate |
| Pd₂(dba)₃ | Bis(dibenzylideneacetone) palladium(0) |
| Ph | Phenyl |
| Phe | Benzenediyl |
| Pye | Pyridinediyl |
| r.t. or RT | room temperature |
| Rac. | Racemic |
| SAM | aminosulfonyl or sulfonamide or SO₂NH₂ |
| SEM | 2-(trimethylsilyl)ethoxymethoxy |
| SPA | scintillation proximity assay |
| Th | 2- or 3-thienyl |
| TFA | trifluoroacetic acid |
| THF | Tetrahydrofuran |
| Thi | Thiophenediyl |
| TLC | thin layer chromatography |
| Tz | 1H (or 2H)-tetrazol-5-yl |
| C₃H₅ | Allyl |

ALKYL GROUP ABBREVIATIONS

| | |
|---|---|
| Me = | Methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | cyclobutyl |
| c-Pen = | cyclopentyl |
| c-Hex = | cyclohexyl |

The present compounds can be prepared according to the general Schemes provided below as well as the procedures provided in the Examples. The following Schemes and Examples further describe, but do not limit, the scope of the invention.

The course of reactions followed in the experimental procedures was followed by thin layer chromatography (TLC) and reaction times are given for illustration only. Melting points are uncorrected and ‘d’ indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. Broad; etc. In addition, “Ar” signifies an aromatic signal. Chemical symbols have their usual meanings; the following abbreviations are used: v (volume), w (weight), b.p. (boiling point), m.p. (melting point), L (liter(s)), ml (milliliters), g (gram(s), mg (milligrams(s), mol (moles), mmol (millimoles), eq (equivalent(s).

### Methods of Synthesis

Compounds of the present invention can be prepared according to the Schemes provided below as well as the procedures provided in the Examples. The substituents are the same as in the above Formulas except where defined otherwise or otherwise apparent to the ordinary skilled artisan.

The novel compounds of the present invention can be readily synthesized using techniques known to those skilled in the art, such as those described, for example, in Advanced Organic Chemistry, March, 4^{th} Ed., John Wiley and Sons, New York, N.Y., 1992; Advanced Organic Chemistry, Carey and Sundberg, Vol. A and B, 3^{rd} Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the present compounds may be prepared using standard synthetic transformations of chemical precursors that are readily available from commercial sources, including Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S.C.); Arcos, (Pittsburgh, Pa.) and Trans World Chemicals (Rockville, Md.).

The procedures described herein for synthesizing the compounds may include one or more steps of protecting group manipulations and of purification, such as, recrystallization, distillation, column chromatography, flash chromatography, thin-layer chromatography (TLC), radial chromatography and high-pressure chromatography (HPLC). The products can be characterized using various techniques well known in the chemical arts, including proton and carbon-13 nuclear magnetic resonance (¹H and ¹³C NMR), infrared and ultraviolet spectroscopy (IR and UV), X-ray crystallography, elemental analysis and HPLC and mass spectrometry (LC-MS). Methods of protecting group manipulation, purification, structure identification and quantification are well known to one skilled in the art of chemical synthesis.

Appropriate solvents are those which will at least partially dissolve one or all of the reactants and will not adversely interact with either the reactants or the product. Suitable solvents are aromatic hydrocarbons (e.g., toluene, xylenes), halogenated solvents (e.g., methylene chloride, chloroform, carbontetrachloride, chlorobenzenes), ethers (e.g., diethyl ether, diisopropylether, tert-butyl methyl ether, diglyme, tetrahydrofuran, dioxane, anisole), nitriles (e.g., acetonitrile, propionitrile), ketones (e.g., 2-butanone, dithyl ketone, tert-butyl methyl ketone), alcohols (e.g., methanol, ethanol, n-propanol, iso-propanol, n-butanol, t-butanol), dimethyl formamide (DMF), dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, sodium carbonate, Cesium carbonate, sodium hydrogen carbonate, and cesium hydrogen carbonate; alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnaesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO.

It is understood that the functional groups present in compounds described in the Schemes below can be further manipulated, when appropriate, using the standard functional group transformation techniques available to those skilled in the art, to provide desired compounds described in this invention.

Other variations or modifications, which will be obvious to those skilled in the art, are within the scope and teachings of this invention. This invention is not to be limited except as set forth in the following claims. 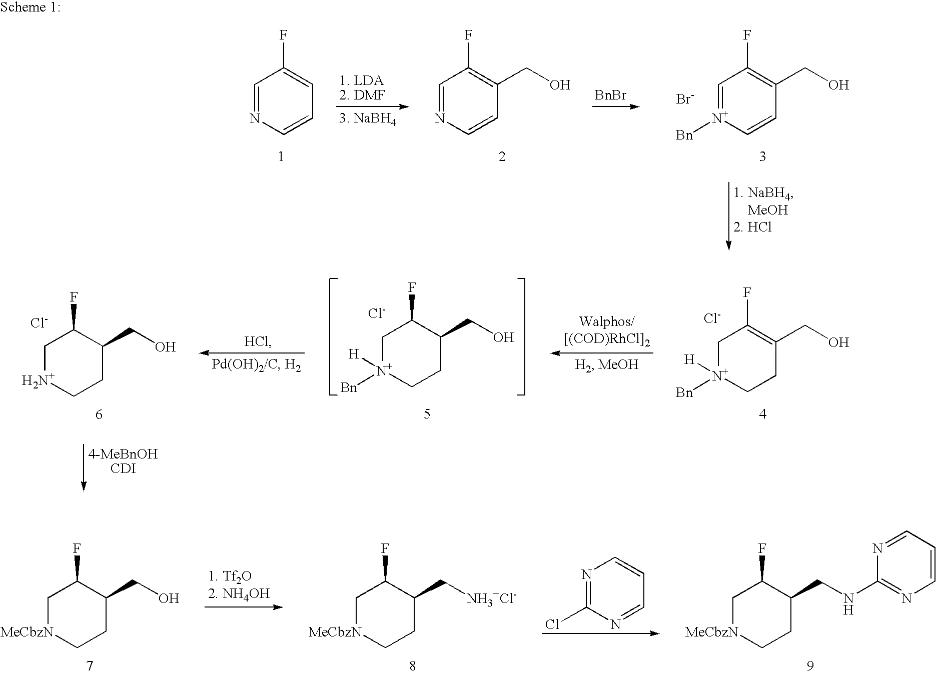 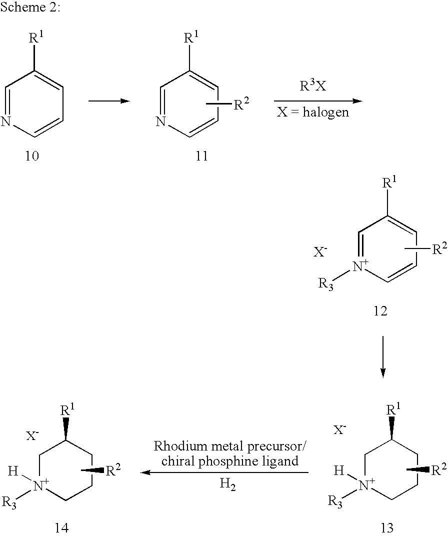
Representative Examples include:

### EXAMPLE 1

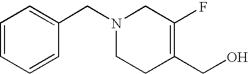 Step A:

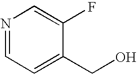

A 5 L round bottom flask was charged with THF (1.87 L, KF≦50 ppm) and cooling to −75° C. was begun. When the temperature of THF had reached ≦−20° C., n-BuLi (11 M in hex, 123 mL) was added over 15 minutes in order to keep the solution temperature below −10° C. When the solution reached −35° C., controlled addition of diisopropylamine (197 mL, KF≦50 ppm) over 15 minutes was carried out so the exotherm did not cause the solution temperature to exceed −16° C. The solution was then allowed to continue to cool until it reached −75° C. 3-Fluoropyridine (compound 1 from Scheme 1) (125 g, KF≦150 ppm) was then added neat to this solution via addition funnel while maintaining the batch temperature below −70° C.

Neat DMF (168 mL, KF≦50 ppm) was then added to the batch over 1 hour maintaining the temperature ≦−70° C. After confirming complete formation of the aldehyde, the reaction was warmed to 0° C., and H₂O (230 mL, 10 eq.) was added. NaBH₄ (48.4 g) was then added in two portions over 5 minutes at 0° C. Addition of concentrated HCl (6 M, 1.17 L) was completed in 1 hour at temperatures between 0-25° C. The reaction batch was then heated to 40° C. and kept at this temperature for 1 hour.

The reaction was then allowed to cool to room temperature. Then, to the aqueous layer 6 M NaOH (747 mL) was slowly added at 0-15° C. to adjust the pH to 12. Approximately 700 mL of H₂O was added to dissolve any precipitate in the aqueous layer. The aqueous layer was then extracted with IPAc (1×1.275 L, 2×800 mL). The organic layer was treated with 20 wt. % Darco-G60 carbon (based on product assay) and the solution was heated to 40° C. for 1 hour followed by filtration over solka floc. After filtration the organic layer was solvent switched from IPAc to IPAc:heptane (15-20% v/v IPAc:heptane). The product crystallized as a white solid. This solution was then cooled to 0° C. for 30 minutes and filtered. An additional 250 mL of heptane was cooled to 0° C. and used to wash the wet cake. Typical Yield=79% (128.5 g).
Step B:

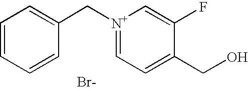

To a 2 L flask under N₂ atmosphere were charged compound 2 from Scheme 1 (50.01 g), acetone (524 mL), and BnBr (50.0 mL). This homogenous solution was heated to reflux for ˜12 h. The reaction mixture was cooled to room temperature and diluted with heptane (550 mL). The pyridinium salt (compound 3 from Scheme 1) was collected by filtration. The wet cake was then slurry washed at ambient temperature with 25% acetone/heptane (200 mL) and filtered. The wet cake was then dried under vacuum at ambient temperature exposed to the atmosphere, affording a slight-pinkish solid ca. 98% pure by ¹H NMR

Typical Yield=93% (109.5 g)

Step C:

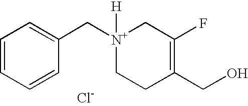

To a 2 L round bottom flask were charged compound 3 from Scheme 1 (100.30 g, 1.00 eq.) and methanol (960 mL). The homogenous solution was then cooled to 10° C. The NaBH₄ (19.10 g, 1.50 Eq) was added portion wise (using a solid addition funnel) while keeping the temperature ≦0° C. The batch was diluted with IPAc (1.0 L), followed by addition of 1 L 11.25 wt % brine. The resulting mixture was aged 15 min, then allowed to separate into two clear layers. The lower brine layer was removed. The organic stream was then washed with 500 mL 15 wt % brine, then allowed to separate into two clear layers. The lower brine layer was removed.

The batch was adjusted to roughly 1:1 MeOH:IPAc (c=100 g/L) and then treated with 25 wt % Ecosorb C-941 at 50° C. in for 2 h. This was then filtered through a plug of celite, while rinsing with 1:1 MeOH:IPAc (rinse was roughly 25% of total batch volume). The batch was then concentrated to a residue.

The batch was then dissolved in 5% MeOH in IPAc at ˜100 g/L (˜636 mL). The batch was warmed to 50° C., followed by addition of a solution of 4M HCl in dioxane (1.10 eq)) slowly over ˜1 h. At this point, the batch was seeded with a small spatula tip full of seed. After complete addition of the HCl solution, the batch was allowed to cool to room temperature slowly overnight. The solids were isolated by filtration. A slurry cake wash was then performed with 5% MeOH/IPAc (200 mL), followed by a displacement wash of 5% MeOH/IPAc (200 mL). The batch was then dried under vacuum at ambient temperature exposed to the atmosphere to afford compound 4 as a white solid (77% yield).

This material, 66.10 g of crude 4, was dissolved in 450 mL MeOH to which was added 450 mL IPAc. This mixture was treated with 25 wt % Ecosorb C-941 (16.53 g) and heated to 50° C. for 2 h. The mixture was then filtered through a pad of celite, washing the Ecosorb C-941 with ˜500 ml 25% MeOH in IPAc. The mixture was then solvent switched on a rotovap to roughly 10% MeOH in IPAc. During the solvent switch, after concentrating to roughly 60% of its original volume, a small spatula tip full of seed was introduced, causing instant crystal growth. This mixture was concentrated until the final volume was ˜350 mL. The slurry was then isolated, using a slurry wash of ˜200 mL 5% MeOH/IPAc. The solids were dried over night under vacuum, exposed to the atmosphere, affording 60.23 g of 4 (70% yield).

Typical Yield=70% (60.2 g).

Step D:

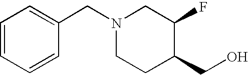

In a N₂ atmosphere glovebox, (R,R)-Walphos (SL-WO03-1) (60.1 mg, commercially available from Solvias, Inc., Fort Lee, N.J. 07024) and [(COD)RhCl]₂ (20.3 mg) were dissolved in dichloromethane (3 mL, anhydrous, N₂ degassed) and aged for 45 min at room temperature. Compound 4 from Scheme 1 (15.0 g) was charged to a 6 oz. glass pressure vessel (Andrews Glass Co., Vineland, N.J.) containing a magnetic stir bar. MeOH (69 mL, anhydrous, N₂ degassed) was added, followed by the catalyst solution and a dichloromethane (3 mL) rinse.

The reactor was degassed with H₂ (40 psig) and immersed in a pre-heated 50° C. oil bath. After a few minutes, the vessel was further pressurized with H₂ to 85 psig and allowed to age for 18.75 h. After this time, the vessel was vented and cooled to room temperature. HPLC analysis indicated >99% conversion of the vinyl fluoride. HPLC analysis indicated 99.3% ee.

The reaction mixture from above was concentrated in vacuo to a dark brown oil, which was then diluted with 50 mL EtOAc, to which was added 50 mL saturated NaHCO₃ (aq). This biphasic mixture was stirred at room temperature for 30 min. This mixture was separated, the aqueous layer was extracted 3×10 mL EtOAc, then the combined organic layers were dried over Na₂SO₄ and concentrated in vacuo to a residue, which was purified by column chromatography (1:1 EtOAc:hexanes) to afford 9.45 g of free base compound 5 (74.4% isolated yield) as a pale yellow oil.

Typical Yield=74% (9.5 g).

Step E:

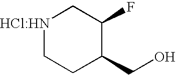

To a 100 mL round bottom flask was charged the free base compound 5 from Example Scheme 1, (1.00 eq), the Pd(OH)₂/C (1.29 g), MeOH (23 mL), and 6M HCl (3.89 mL, 1.00 eq.). This mixture was degassed three times, finally filling the vessel with H₂ (1 atm, balloon pressure). The reaction was stirred at room temperature for 18 h. The mixture was filtered through a plug of Celite 521, rinsed with 50 mL MeOH, then concentrated to a residue. The residue was redissolved in 150 mL 1:1 MeOH:IPAc, then refiltered through a sintered glass funnel to remove inorganics. This resulting solution was then solvent switched to roughly 10% MeOH in IPAc, during which spontaneous crystallization of compound 6 from Scheme 1 was observed. The solids were isolated by vacuum, washed twice with ˜10 mL 10% MeOH in IPAc, then dried under vacuum over night, affording a pale white, crystalline solid.

Typical Yield=81% (3.2 g).

Step F:

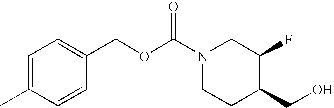

N,N′-Carbonyldiimidazole, 2.39 g (1.00 eq) was charged to a 50 mL round bottom flask, to which was added the DMF (19.7 ml). Then, the 4-methylbenzyl alcohol (1.80 g 1.00 eq) was added as a solid. This mixture was stirred for 15 min. at room temperature, during which an exotherm was noted (ΔT=+6.1° C., 18.5° C. to 24.6° C.). The fluoroalcohol HCl salt 6, 2.50 g (1.00 eq) was then added as a solid to this mixture. This was heated to 50° C. for 10 h, and then allowed to cool to room temperature over night. The resulting mixture was diluted with 40 mL EtOAc. This mixture was washed 2×25 mL 3M HCl and separated, then 1×25 mL 15 wt % brine and separated. This was extracted with 1×15 mL EtOAc and combined with the previous organic stream. The organic stream was concentrated to a residue and subjected to column chromatography eluting with a gradient (0% to 50% EtOAc in hexanes, TLC's developed in 50% EtOAc:hexanes, visualizing with UV and KMnO₄), to afford 3.35 g of a clear colorless oil.

Typical Yield=81% (3.4 g).

Step G:

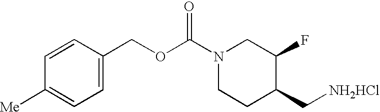

A solution of fluoro alcohol compound 7 from Scheme 1 (1.22 g) in CH₃CN was cooled to −20° C. and Hunig's base (2.2 equiv., 1.66 mL) was added. To this, Tf₂O—(1.1 equiv., 0.81 mL) was slowly added while maintaining the internal temperature ≦−10° C. Aqueous NH₄OH (15 equiv., 2.7 mL) was then added to the reaction mixture at low temperature (−20° C.) and then warmed up to room temperature and aged for 1 h. After completion, toluene (15 mL) and 10% NaOH (10 mL) were added and the layers separated. After extraction, the organic layer was washed with H₂O (10 mL).

The toluene stream of the amine was dried (˜400 μg/mL) and concentrated to 100 g/L. Methanol was then added to obtain an overall solvent composition of toluene/MeOH (95:5), followed by the slow addition of HCl (1.05 equiv, 1.12 ml) at 50° C. The amine hydrochloride 8 from Scheme 1 crystallized immediately, and the reaction was aged 20 min. The light yellow salt was then filtered and washed with cold toluene (15 mL) to offer amine hydrochloride 8 in 82% as a white crystalline solid.
Step H:

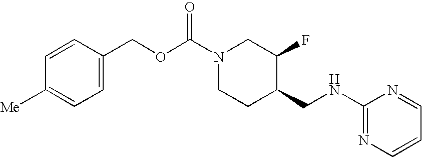

Into a 100-L round bottom flask were charged 1.67 kg amine HCl salt 8 from Scheme 1, 912.4 g chloropyrimidine, 4.6 L of diisopropylethyl amine and 25.78 L ethylene glycol. The resulting slurry gradually became a solution, which was degassed and stirred under a nitrogen atmosphere. The contents were heated to 100° C. for 12 h. The heat was turned off and the reaction solution slowly cooled to room temperature, which resulted in the formation of a slurry. To the slurry was added 77.3 L water over 1 h period and the slurry was aged at room temperature for 3 h. The mixture was filtered and the cake was washed with additional 80 L. The wet cake was left under nitrogen to dry overnight. After drying, 1.90 kg of an off white solid was collected.

1.77 kg of the above solid was dissolved into 71 L EtOAc and treated with 531 g Darco G-60 carbon at room temperature for 3 h. Filtration through Solka Floc was followed by washing with 2×20 L EtOAc. A solvent switch to MeOH under reduced pressure resulted in a slurry, and the final MeOH volume was adjusted to 19 L. The slurry in MeOH was heated to ca. 60° C. Gradually cooling to room temperature resulted in a slurry, to which 57 L GMP water was added over 1 h with cooling (exothermic mixing, temperature controlled below 30° C.). The mixture was aged at room temperature for 3 h and filtered to collect solid, the cake was washed with 30 L GMP water and left to dry under nitrogen. 1.55 kg dried product was collected. (89% yield).

Typical Yield=89% (1.55 kg).

The following Examples 2-7 can be prepared using intermediates and procedures described above.

### EXAMPLE 2

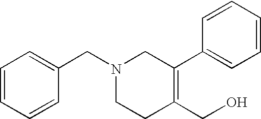

### EXAMPLE 3

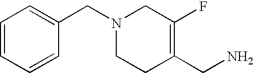

### EXAMPLE 4

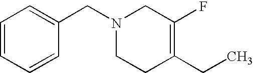

### EXAMPLE 5

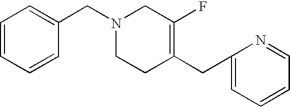

### EXAMPLE 6

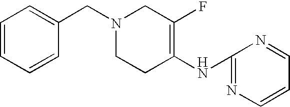

### EXAMPLE 7

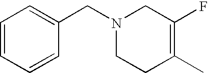

### EXAMPLE 8

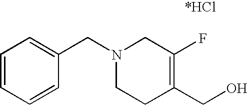

What is claimed is:

1. A process for preparing a compound of Formula I: 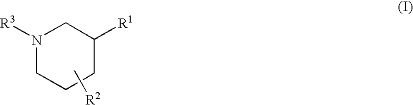
or a pharmaceutically acceptable salt thereof,
wherein
R¹ is halogen, oxygen, CONH₂, nitrogen, sulfur, silicon, optionally substituted C₁-C₆ alkyl or optionally substituted aryl;
R² is oxygen, amino, halogen, CONH₂, nitrogen, sulfur, or C₀-C₄ alkyl optionally substituted with one or more groups selected from hydrogen, hydroxy, amino, and amino-heteroaryl;
R³ is sulfur, optionally substituted C₁-C₆ alkyl, aryl, phosphorous, silicon, benzyl, CBZ, carbamate,
C₁-C₆alkyl-optionally substituted aryl, or C(═O)O-optionally substituted aryl;
the process comprising an asymmetric reduction of a compound of Formula (II): 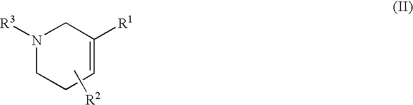
wherein
R¹, R² and R³ each is as defined above,
in a suitable organic solvent in the presence of a metal precursor complexed to a chiral mono- or bisphosphine ligand.

2. The process of claim 1 wherein said chiral monophosphine ligand is of the structural formula: 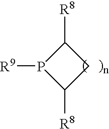
wherein n is 1, 2, or 3; R⁸ is C₁₋₈ alkyl or C₆₋₁₀ aryl; and R⁹ is aryl or a ferrocenyl phospholane radical.

3. The process of claim 2 wherein R⁹ is phenyl and R⁸ is C₁₋₄ alkyl or aryl.

4. The process of claim 2 wherein said chiral phosphine ligand is of the structural formula: 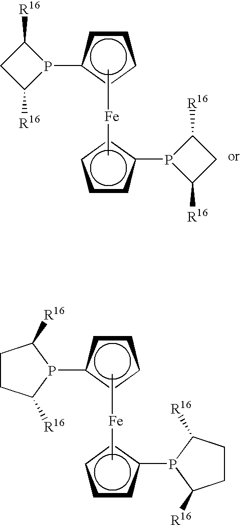
wherein R¹⁶ is C₁₋₄ alkyl or aryl;
or the corresponding enantiomers thereof.

5. The process of claim 1 wherein said chiral bisphosphine ligand is of the following structural formula: 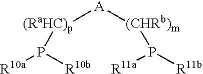
wherein m and p are each 0 or 1;
R^{a} and R^{b} are each independently hydrogen, C₁₋₄ alkyl, or C₃₋₆ cycloalkyl;
A represents (a) a C₁₋₅ alkylene bridge optionally containing one to two double bonds said C₁₋₅ alkylene bridge being unsubstituted or substituted with one to four substituents independently selected from the group consisting of C₁₋₄ alkyl, C₁₋₄ alkoxy, aryl, and C₃₋₆ cycloalkyl and said C₁₋₅ alkylene bridge being optionally fused with two C₅₋₆ cycloalkyl, C₆₋₁₀ aryl, or C₆₋₁₀ heteroaryl groups unsubstituted or substituted with one to four substituents independently selected from the group consisting of C₁₋₄ alkyl, C₁₋₄ alkoxy, chloro, and fluoro; (b) a 1,2-C₃₋₈ cycloalkylene bridge optionally containing one to three double bonds and one to two heteroatoms selected from NC₀₋₄ alkyl, N(CH₂)₀₋₁Ph, NCOC₁₋₄ alkyl, NCOOC₁₋₄ alkyl, oxygen, and sulfur and said 1,2-C₃₋₈ cycloalkylene bridge being unsubstituted or substituted with one to four substituents independently selected from the group consisting of C₁₋₄ alkyl, C₁₋₄ alkoxy, oxo, aryl, and C₃₋₆ cycloalkyl; (c) a 1,3-C₃₋₈ cycloalkylene bridge optionally containing one to three double bonds and one to two heteroatoms selected from NC₀₋₄ alkyl, N(CH₂)0-1Ph, NCOC₁₋₄ alkyl, NCOOC₁₋₄ alkyl, oxygen, and sulfur and said 1,3-C₃₋₈ cycloalkylene bridge being unsubstituted or substituted with one to four substituents independently selected from the group consisting of C₁₋₄ alkyl, C₁₋₄ alkoxy, oxo, aryl, and C₃₋₆ cycloalkyl; or (d) 1,2-phenylene unsubstituted or substituted with one to three substituents independently selected from halogen, C₁₋₄ alkyl, hydroxy, and C₁₋₄ alkoxy; and R^{10a}, R^{10b}, R^{11a}, and R^{11b} are each independently C₁₋₆ alkyl, C₃₋₆ cycloalkyl, or aryl with alkyl, cycloalkyl, and aryl being unsubstituted or substituted with one to three groups independently selected from the group consisting of C₁₋₄ alkyl, C₁₋₄ alkoxy, chloro, and fluoro; or R^{10a} and R^{10b} when taken together or R^{11a} and R^{11b} when taken together can form a 4- to 7-membered cyclic aliphatic ring unsubstituted or substituted with two to four substituents independently selected from the group consisting of C₁₋₄ alkyl, C₁₋₄ alkoxy, hydroxymethyl, C₁₋₄ alkoxymethyl, aryl, and C₃₋₆ cycloalkyl and said cyclic aliphatic ring being optionally fused with one or two aryl groups.

6. The process of claim 5 wherein R^{10a} and R^{10b} represent the same substituent which are both structurally distinct from R^{11a} and R^{11b} which represent the same but structurally distinct substituent.

7. The process of claim 5 wherein said chiral bisphosphine ligand is of the structural formula: 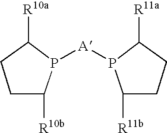
wherein A′ is CH₂; CH₂CH₂; 1,2-phenylene; 2,5-furandione-3,4-diyl; or N-methyl-2,5-pyrroledione-3,4-diyl; and R^{10a}, R^{10b}, R^{11a}, and R^{11b} are each independently C₁₋₄ alkyl, C₁₋₄ alkoxy, CH₂OH, or CH₂OC₁₋₄ alkyl.

8. The process of claim 1 wherein said chiral bisphosphine ligand is of the structural formula: 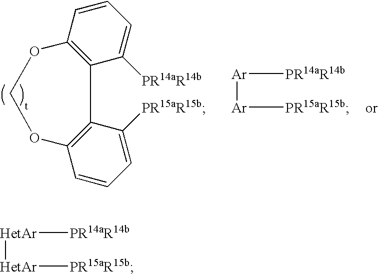
wherein t is an integer from one to six;
Ar is phenyl or naphthyl unsubstituted or substituted with one to four substituents independently selected from C₁₋₄ alkyl, C₁₋₄ alkoxy, chloro, and fluoro; or two adjacent substituents on Ar together with the carbon atoms to which they are attached form a five-membered methylenedioxy ring;
HetAr is pyridyl or thienyl each of which is unsubstituted or substituted with one to four substituents independently selected from C₁₋₄ alkyl, C₁₋₄ alkoxy, chloro, and fluoro; or two adjacent substituents on HetAr together with the carbon atoms to which they are attached form a five-membered methylenedioxy ring;
R^{14a}, R^{14b}, R^{15a}, and R^{15b} are each independently C₁₋₄ alkyl, aryl, or C₃₋₆ cycloalkyl wherein aryl and cycloalkyl are unsubstituted or substituted with one to four substituents independently selected from C₁₋₄ alkyl and C₁₋₄ alkoxy; or
or R^{14a} and R^{14b} when taken together or R^{15a} and R^{15b} when taken together can form a 4- to 7-membered cyclic aliphatic ring unsubstituted or substituted with two to four substituents independently selected from the group consisting of C₁₋₄ alkyl, C₁₋₄ alkoxy, hydroxymethyl, C₁₋₄ alkoxymethyl, aryl, and C₃₋₆ cycloalkyl and said cyclic aliphatic ring being optionally fused with one or two aryl groups.

9. The process of claim 8 wherein R^{14a} and R^{14b} represent the same substituent which are both structurally distinct from R^{15a} and R^{15b} which represent the same but structurally distinct substituent.

10. The process of claim 8 wherein said chiral bisphosphine ligand is of the structural formula: 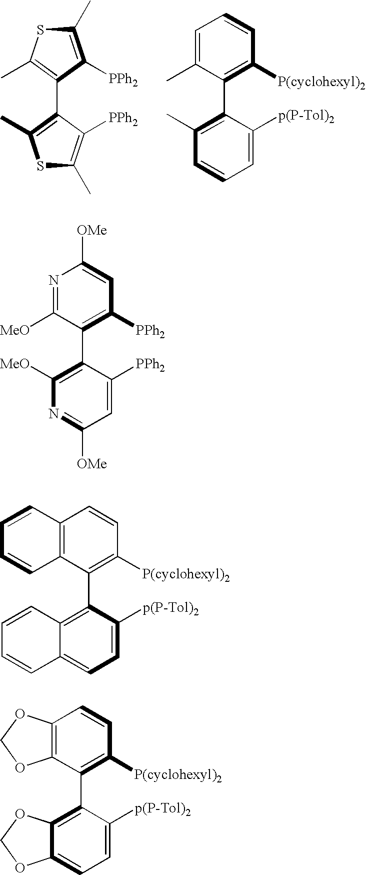
or the corresponding enantiomers thereof.

11. The process of claim 5 wherein said chiral bisphosphine ligand is of the structural formula: 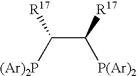
wherein Ar is aryl and R¹⁷ is C₁₋₄ alkyl or aryl;
or the corresponding enantiomers thereof;
with the proviso that when Ar is unsubstituted phenyl, then R¹⁷ is not methyl.

12. The process of claim 1 wherein said chiral bisphosphine ligand is of the structural formula: 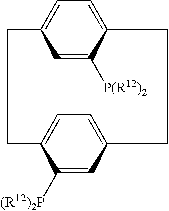
wherein R¹² is C₁₋₄ alkyl, C₃₋₆ cycloalkyl, or aryl;
or the corresponding enantiomers thereof.

13. The process of claim 12 wherein aryl is phenyl.

14. The process of claim 1 wherein said chiral bisphosphine ligand is of the structural formula: 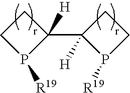
wherein r is 1, 2, or 3; and R¹⁹ is C₁₋₄ alkyl or aryl;
or the corresponding enantiomers thereof.

15. The process of claim 1 wherein said chiral bisphosphine ligand is a ferrocenyl bisphosphine ligand of the structural formula: 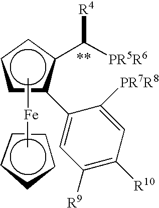
wherein ** is a carbon stereogenic center with an (R)-configuration;
R⁴ is C₁-C₄ alkyl or aryl;
R⁵, R⁶, R⁷ and R⁸ are each independently C₁-C₆ alkyl, C₅₋₁₂ cycloalkyl, heteroaryl or aryl, wherein said aryl and heteroaryl is optionally substituted with one or more C₁-C₆ fluoroalkyl, halogen, C₁-C₄ alkyl, CF₃, or O—C₁-C₄ alkyl; and R⁹ and R¹⁰ are each independently halogen, hydrogen, C₁-C₆ alkyl, C₁-C₆ fluoroalkyl, C₅-C₁₂ cycloalkyl or C₁-C₄ alkoxy.

16. The process of claim 15 wherein R⁴ is methyl; R⁵ and R⁶ are each independently cyclohexyl; R⁷ and R⁸ are each independently phenyl; and R⁹ and R¹⁰ are each independently hydrogen.

17. The process of claim 15 wherein said metal precursor is [Rh(cod)Cl]₂.

18. The process of claim 15 wherein said organic solvent is methanol.

19. The process of claim 1 wherein said chiral bisphosphine ligand is a ferrocenyl bisphosphine ligand of the structural formula: 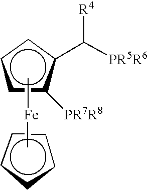
wherein R⁴ is C₁₋₄ alkyl or aryl; and
R⁵, R⁶, R⁷ and R⁸ are each independently C₁-C₆ alkyl, C₅₋₁₂ cycloalkyl, heteroaryl or aryl, wherein said aryl and heteroaryl is optionally substituted with one or more C₁-C₆ fluoroalkyl, halogen, C₁-C₄ alkyl, CF₃, or O—C₁-C₄ alkyl.

20. The process of claim 1 wherein said chiral monophosphine ligand is of the structural formula: 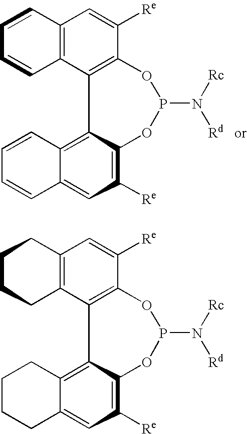
wherein R^{e} is hydrogen or methyl; R^{c} and R^{d} are each independently hydrogen, C₁₋₄ alkyl, benzyl, or α-methylbenzyl; or R^{c} and R^{d} together with the nitrogen atom to which they are attached form a pyrrolidine or piperidine ring.

21. An intermediate compound represented by 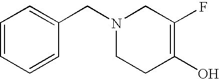
or an organic acid or metal acid thereof.
